SEQUENCE LISTING (6 sequences)

SEQ ID NO: 1 - protein, 9 aa, Artificial
  synthetic peptide
WAGGDASGE

SEQ ID NO: 2 - protein, 11 aa, Artificial
  synthetic peptide
MHRQETVDCLK

SEQ ID NO: 3 - protein, 12 aa, Artificial
  synthetic peptide
TRDIYETDYYRK

SEQ ID NO: 4 - protein, 16 aa, Artificial
  synthetic peptide
AAKIQASFRGHMARKK

SEQ ID NO: 5 - protein, 7 aa, Artificial
  synthetic peptide
RVYIHPF

SEQ ID NO: 6 - protein, 11 aa, Artificial
  synthetic peptide
DAEFRHDSGYE

(19) United States
(12) Patent Application Publication
(10) Pub. No.: US 2009/0142851 A1
(43) Pub. Date: Jun. 4, 2009

(54) METHOD FOR SELECTIVELY RECOVERING C-TERMINAL PEPTIDE OF PROTEIN AND METHOD FOR DETERMINING AMINO ACID SEQUENCE OF C-TERMINAL PEPTIDE OF PROTEIN USING THE SAME

(76) Inventors: Keisuke Shima, Kyoto-shi (JP); Minoru Yamaguchi, Kyoto-shi (JP); Hiroki Kuyama, Kyoto-shi (JP); Eiji Ando, Kyoto-shi (JP); Osamu Nishimura, Kawanishi-shi (JP); Susumu Tsunasawa, Otsu-shi (JP); Kazuhiro Sonomura, Kyoto-shi (JP)

Correspondence Address: Cheng Law Group, PLLC, 1100 17th Street, N.W., Suite 503, Washington, DC, 20036, US

(21) Appl. No.: 12/325,115

(22) Filed: Nov. 28, 2008

(30) Foreign Application Priority Data
Nov. 30, 2007 (JP) 2007-310218

(51) Int. Cl. G01N 33/68; C07K 1/14; C12P 21/06

(52) U.S. Cl. 436/89; 530/344; 435/68.1

(57) ABSTRACT

The present invention provides a method for specifically recovering a C-terminal peptide fragment, and a method for easily determining the sequence of a C-terminal peptide fragment, which is difficult to be determined by a conventional method, with the use of a mass spectrometer, in particular a method capable of de novo sequencing of a C-terminal peptide fragment. A method for selectively recovering a C-terminal peptide of a protein, comprising the steps of: in a cleavage product of a protein containing a C-terminal peptide fragment (A) having an α-amino group but not having an ε-amino group and the other peptide fragments (B) having an α-amino group and an ε-amino group, selectively modifying the α-amino groups to obtain a C-terminal peptide fragment modified (A′) and the other peptide fragments modified (B′); and separating the C-terminal peptide fragment modified (A′) from the modified cleavage product by allowing a carrier to hold the other peptide fragments modified (B′) via the ε-amino group. A method for determining the amino acid sequence of a C-terminal peptide of a protein, comprising the steps of: selectively recovering a C-terminal peptide of a protein by the above method; and determining the amino acid sequence by subjecting a recovered C-terminal peptide fragment to mass spectrometry measurement.

### BACKGROUND OF THE INVENTION

1. Field of the Invention

The present invention relates to the field of determining the amino acid sequence of a protein. More specifically, the present invention relates to a method for selectively recovering a C-terminal peptide of a protein and a method for determining the amino acid sequence of a C-terminal peptide of a protein using the same.

2. Disclosure of the Related Art

As a conventional method for recovering a C-terminal portion of a protein, there is a method in which peptides obtained by digesting a protein with lysyl endopeptidase are coupled to p-phenylenediisothiocyanate (DITC) glass via their ε-amino groups, and then the coupled peptides are subjected to cleavage with trifluoroacetic acid (TFA) to specifically recover a C-terminal peptide fragment not having an ε-amino group (Japanese Patent Application Laid-open No. H1-235600).

Further, as a method for de novo sequence analysis using a protein mass spectrometer, there is a method in which a peptide mixture obtained by tryptic digestion of a protein is reacted with a tris(2,4,6-trimethoxyphenyl)phosphonium acetic acid N-hydroxysuccinimide ester (TMPP-Ac-Osu) to obtain N-terminally TMPP-derivatized peptides, and then the peptides are separated by HPLC and analyzed by MALDI-TOFMS (Analytical Biochemistry 268, 305-317 (1999)).

On the other hand, as a method for recovering an N-terminal fragment of a protein for de novo sequence analysis, there is a method in which side-chain amino groups of amino acid residues of a protein are protected, and then the protein is enzymatically digested to obtain one kind of N-terminal peptide fragment derived from the N-terminal of the protein and the other peptide fragment(s), and then the N-terminal peptide fragment is separated from the other peptide fragment(s) using a DITC resin to recover the N-terminal peptide (Japanese Patent Application Laid-open No. 2004-219412).

### SUMMARY OF THE INVENTION

In the case of the method disclosed in Japanese Patent Application Laid-open No. H1-235600, since a strong acid TFA is used, it is difficult to manually recover. This makes it difficult to provide a reagent kit, and therefore it is necessary to develop an apparatus capable of automatically carrying out this method.

In general, when a protein is digested with an enzyme which cleaves peptide bonds on the C-terminal side of a specific amino acid (e.g., trypsin), the C-terminal amino acids of digested peptides can be almost completely identified. Further, when a protein is digested with trypsin or lysyl endopeptidase, the C-terminal amino acids of digested peptides (more specifically, an N-terminal fragment and internal fragment(s)) are positively charged, and therefore high detection sensitivity can be achieved in mass spectrometry measurement.

However, in the case of using the method disclosed in Japanese Patent Application Laid-open No. H1-235600, even when a protein is digested with such an enzyme, the kind of the C-terminal amino acid of the protein cannot be identified and the C-terminal amino acid is not always positively charged. Therefore, the detection sensitivity of the C-terminal peptide of a protein recovered by this method is lower than that of the other digested peptides. Further, since the C-terminal amino acid of a protein cannot be identified, it is difficult to determine the sequence of the C-terminal peptide by mass spectrometry measurement. Further, also in a case where the sequence of the C-terminal peptide of a protein is determined using a protein sequencer, there is a limit on sensitivity as compared to a case using a mass spectrometer.

However, in a case where digested peptides of a protein are subjected to mass spectrometry measurement without recovering a C-terminal peptide, the C-terminal peptide is indistinguishable in the digested peptides. In this case, the internal sequence of the protein can be determined, but the sequence of a C-terminal portion of the protein cannot be determined.

Analytical Biochemistry 268, 305-317 (1999) only discloses that the de novo sequence analysis of an N-terminally TMPP-derivatized protein enzymatic digest has become possible, but according to this document, a peptide fragment containing a C-terminal portion of a protein is indistinguishable in the peptide fragments contained in the N-terminally TMPP-derivatized protein enzymatic digest. Therefore, the internal sequence of the protein can be determined, but the sequence of the C-terminal portion cannot be determined.

In the case of the method disclosed in Japanese Patent Application Laid-open No. 2004-219412, since the use of an N-terminal labeling reagent causes also the modification of side-chain amino groups, it is absolutely necessary to previously protect side-chain amino groups.

It is therefore an object of the present invention to provide a method for specifically recovering a C-terminal peptide fragment and a method for easily determining the sequence of a C-terminal peptide fragment, which is difficult to be determined by a conventional method, with the use of a mass spectrometer. More particularly, it is an object of the present invention to provide a method capable of de novo sequencing of a C-terminal peptide fragment.

The present inventors have extensively studied, and as a result, have found that the above object can be achieved by TMPP modification of a lysyl endopeptidase digest. This finding has led to the completion of the present invention.

The present invention includes the following.
(1) A method for selectively recovering a C-terminal peptide of a protein, comprising the steps of:

providing a cleavage product of a protein of interest containing a C-terminal peptide fragment (A) having an α-amino group but not having an ε-amino group and the other peptide fragments (B) having an α-amino group and an ε-amino group;

selectively modifying the α-amino groups in the cleavage product of the protein of interest with a modification reagent to obtain a modified cleavage product containing a C-terminal peptide fragment modified (A′) having a modified amino group but not having the ε-amino group and the other peptide fragments modified (B′) having a modified amino group and the ε-amino group; and

separating the C-terminal peptide fragment modified (A′) from the modified cleavage product by allowing a carrier to hold the other peptide fragments modified (B′) via the ε-amino group.
(2) The method for selectively recovering a C-terminal peptide of a protein according to the above (1), wherein the modification reagent is positively-charged.

The method according to the above (2) is useful in a case where the recovered C-terminal peptide is to be subjected to mass spectrometry. More specifically, since the N-terminal of a C-terminal peptide to be recovered is given a positive charge, it is possible to improve the sensitivity of a modification group-containing ion species among fragment ion species generated in mass spectrometry, especially in MS/MS analysis such as PSD or CID. As a result, the complexity of fragment ions observed is reduced, thereby facilitating amino acid sequence analysis.
(3) The method for selectively recovering a C-terminal peptide of a protein according to the above (1) or (2), wherein the modification reagent is selected from the group consisting of tris(2,4,6-trimethoxyphenyl)phosphonium acetic acid and derivatives thereof.

Examples of the derivatives of tris(2,4,6-trimethoxyphenyl)phosphonium acetic acid include esters, active esters, acid halides, acid anhydrides, and acid azides of tris(2,4,6-trimethoxyphenyl)phosphonium acetic acid.
(4) The method for selectively recovering a C-terminal peptide of a protein according to any one of the above (1) to (3), wherein the modification reagent is selected from the group consisting of a tris(2,4,6-trimethoxyphenyl)phosphonium acetic acid N-hydroxysuccinimide ester and a tris(2,4,6-trimethoxyphenyl)phosphonium acetic acid sulfosuccinimide ester.

The method according to the above (3) or (4) makes it possible to easily and effectively carry out selective modification of α-amino groups in the cleavage product of a protein of interest.
(5) The method for selectively recovering a C-terminal peptide of a protein according to any one of the above (1) to (4), wherein the cleavage product of the protein of interest is obtained by digesting a protein of interest with lysyl endopeptidase.
(6) The method for selectively recovering a C-terminal peptide of a protein according to any one of the above (2) to (5), further comprising the step of chemically modifying a side chain of an arginine residue that can be contained in the cleavage product of the protein of interest, the modified cleavage product, or the separated C-terminal peptide fragment (A′).

The method according to the above (6) is useful in a case where the recovered C-terminal peptide is to be subjected to mass spectrometry. More specifically, in the case where the recovered C-terminal peptide is to be subjected to mass spectrometry, modification of a side chain of an arginine residue may be carried out at any time before the step of mass spectrometry. Such modification makes it possible to cancel the electric charge of a side chain of an arginine residue, thereby promoting fragmentation in MS/MS and facilitating sequence analysis.
(7) The method for selectively recovering a C-terminal peptide of a protein according to any one of the above (1) to (6), wherein the carrier has p-phenylenediisothiocyanate immobilized thereto.
(8) A method for determining the amino acid sequence of a C-terminal peptide of a protein, comprising the steps of:

selectively recovering a C-terminal peptide of a protein of interest by the method according to any one of the above (1) to (7); and

determining the amino acid sequence of the C-terminal peptide by subjecting a recovered C-terminal peptide fragment to mass spectrometry measurement.

According to the present invention, it is possible to specifically recover a C-terminal peptide fragment and to easily determine the sequence of a C-terminal peptide fragment, which is difficult to be determined by a conventional method, with the use of a mass spectrometer. Particularly, according to the present invention, de novo sequencing of a C-terminal peptide fragment becomes possible.

### BRIEF DESCRIPTION OF THE DRAWINGS

FIG. 1 shows an MS spectrum of a product obtained by TMPP modification of a mixture of four kinds of model peptides in Experimental Example 1;

FIG. 2 shows an MS spectrum of a peptide isolated by subjecting the product obtained by TMPP modification of a mixture of four kinds of model peptides to separation using a DITC resin in Experimental Example 1;

FIG. 3 shows an MS spectrum of a Lys-C digest of lysozyme obtained in Example 1;

FIG. 4 shows an MS spectrum of a product obtained by TMPP modification of a Lys-C digest of lysozyme in Example 1;

FIG. 5 shows an MS spectrum of a C-terminal peptide fragment isolated by subjecting the product obtained by TMPP modification of a Lys-C digest of lysozyme to separation using a DITC resin in Example 1;

FIG. 6 shows a result of amino acid sequencing of the C-terminal peptide fragment isolated in Example 1 by MS/MS (CID);

FIG. 7 shows a CID spectrum of a product obtained by TMPP modification of a model peptide in Experimental Example 2;

FIG. 8 shows a CID spectrum of a product obtained by TMPP modification and modification of an arginine residue of a model peptide in Experimental Example 2;

FIG. 9 shows a CID spectrum of the product obtained by TMPP modification of a model peptide in Experimental Example 2; and

FIG. 10 shows a CID spectrum of the product obtained by TMPP modification and modification of an arginine residue of a model peptide in Experimental Example 2.

### DETAILED DESCRIPTION OF THE INVENTION

### <1. Cleavage Product of Protein of Interest>

According to the present invention, first, a cleavage product of a protein of interest is provided.

The cleavage product in the present invention contains a peptide fragment, as a C-terminal peptide fragment (A), having an α-amino group but not having an ε-amino group, and peptide fragments, as the other peptide fragments (B), having both an α-amino group and an ε-amino group. Here, the other peptide fragments include an N-terminal peptide fragment and internal peptide fragment(s).

Such a protein cleavage product may be prepared by cleaving a protein of interest by a method for cleaving peptide bonds on the C-terminal side of lysine residues. As such a method, one well known to those skilled in the art may be appropriately used.

Specific examples of such a method include digestion using lysyl endopeptidase. The lysyl endopeptidase is not particularly limited as long as it may specifically cleave peptide bonds on the C-terminal side of lysine residues. Examples of the lysyl endopeptidase include Lys-C and API.

Alternatively, the cleavage product of the present invention may be prepared using an enzyme other than the lysyl endopeptidase. For example, since trypsin specifically cleaves peptide bonds on the C-terminal side of lysine and arginine residues, the cleavage product of the present invention can be prepared by chemically modifying arginine residues of a protein of interest and then digesting the protein of interest with trypsin.

In a case where the protein cleavage product is prepared using lysyl endopeptidase, a side chain of an arginine residue of peptide fragments contained in a sample is preferably modified. In this case, modification of a side chain of an arginine residue may be carried out at any time before a mass spectrometry step, which will be described later, is carried out. By carrying out such modification, the protonation degree of a side chain of an arginine residue is lowered so that a side chain of an arginine residue is less likely to be positively charged. As a result, it is possible to more effectively obtain the effect of promoting the generation of fragment ions (which will be described later) in mass spectrometry (PSD, CID).

A method for modifying a side chain of an arginine residue is not particularly limited, and may be appropriately determined by those skilled in the art. Examples of such a modification method include a method using 2,3-butanedione (which may be carried out with reference to, for example, Anal. Chim. Acta, 528, 165-173 (2005)), a method using 1,1,1,5,5,5-hexafluoro-2,4-pentanedione (which may be carried out with reference to, for example, Int. J. Mass Spectrom. Ion Proc. 169/170, 127-140 (1997)), a method using cyclohexane-1,2-dione (which may be carried out with reference to, for example, J. Biol. Chem., 242, 1036 (1967)), a method using acetylacetone (which may be carried out with reference to, for example, J. Mass Spectrom. 32, 1337-1349 (1997)), and a method using malondialdehyde (which may be carried out with reference to, for example, J. Mass Spectrom., 41, 623-632 (2006)).

On the other hand, in the case of carrying out a modification reaction of a side chain of an arginine residue using acetylacetone, the reaction temperature is also preferably set to 75 to 85° C. (e.g., about 80° C.). By carrying out the modification reaction under such temperature conditions in the present invention, it is possible to obtain a target material in good yield in a short time. Under such temperature conditions, the reaction time may be set to, for example, 2 to 4 hours (e.g., about 3 hours).

The following scheme 1 shows an embodiment in which a protein of interest is subjected to the preparation of Lys-C digest by Lys-C protease digestion to give a C-terminal peptide fragment (A) and the other peptide fragments (B). In the scheme 1, a to 1 each represents an amino acid residue other than a lysine residue, and a portion represented by Lys-NH₂ is a lysine residue.

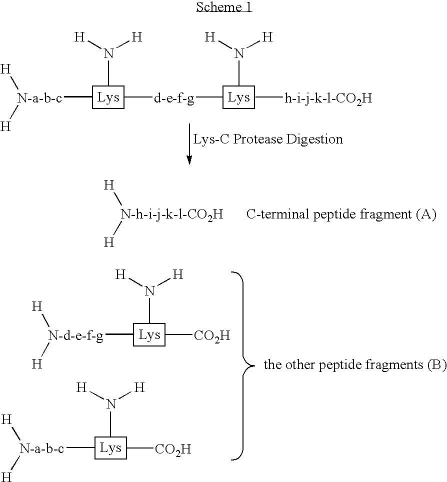

### <2. Modification Step>

The protein cleavage product is subjected to a modification step. In the modification step, α-amino groups of the C-terminal peptide fragment (A) and the other peptide fragments (B) are selectively modified, but on the other hand, ε-amino groups of the other peptide fragments (B) are not modified.

A modification reagent to be used in the modification step is preferably an electrically-charged (e.g., positively-charged) modification reagent.

By using an electrically-charged modification reagent, it is possible to give an electric charge to the terminal of each peptide fragment. In a case where such a modified peptide fragment is subjected to mass spectrometry, especially MS/MS analysis such as PSD or CID, the sensitivity of a modification group-containing ion species among fragment ions generated by fragmentation can be increased (in a case where a positively-charged modification reagent is used) or reduced (in a case where a negatively-charged modification reagent is used) As a result, it is possible to reduce the complexity of the fragment ions observed, thereby facilitating amino acid sequence analysis. As described above, the use of an electrically-charged modification reagent is preferred in that it facilitates the amino acid sequence analysis of an obtained modified peptide fragment by mass spectrometry.

Examples of the modification reagent include appropriately determined by those skilled in the art. For example, such selective modification may be carried out with reference to Rapid Commun. Mass Spectrom. 12, 603-608 (1998) or Proteomics 2004, 4, 1684-1694.

Other examples of the modification reagent include fluorescence dyes having a modification group such as a tetrafluorophenyl (TFP) esters, an isothiocyanates, a sulfonyl chlorides, a dichlorotriazines, 4-sulfo-2,3,5,6-tetrafluorophenol (STP), or a succinimide ester of sulfodichlorophenyl (SDP). Examples of such fluorescence dyes include Alexa Fluor®, BODIPY®, fluorescein, tetramethylrhodamine, rhodamine, and Texas Red®. A method for selectively modifying α-amino groups with such a fluorescence dye is not particularly limited, and may be appropriately determined by those skilled in the art. In the case of using such a fluorescence dye, by adjusting pH during the modification reaction within a neutral region, it is possible to enhance α-amino group selectivity for modification.

Further, an isocyanate-coupled resin may also be used as a modification reagent. A method for selectively modifying α-amino groups with such a resin is not particularly limited, and may be appropriately determined by those skilled in the art. Modification with such a resin may be carried out with reference tris(2,4,6-trimethoxyphenyl)phosphonium acetic acid and its derivatives. Examples of the derivatives of tris(2,4,6-trimethoxyphenyl)phosphonium acetic acid include esters, active esters, acid halides, acid anhydrides, and acid azides and the like of tris(2,4,6-trimethoxyphenyl)phosphonium acetic acid. Examples of the active esters of tris (2,4, 6-trimethoxyphenyl)phosphonium acetic acid include a tris(2,4,6-trimethoxyphenyl)phosphonium acetic acid N-hydroxysuccinimide ester (TMPP-Ac-OSu) and a tris(2,4,6-trimethoxyphenyl)phosphonium acetic acid sulfosuccinimidyl ester. More specific examples of the modification reagent include (succinimidyloxycarbonylmethyl)tris(2,4,6-trimethoxyphenyl)phosphonium bromide. By using such a modification reagent, it is possible to easily and effectively carry out selective modification of α-amino groups in the cleavage product of a protein of interest.

In addition to the above-mentioned modification reagents, the following modification reagents may also be used. For example, a 5-bromonicotinic acid N-hydroxysuccinimide ester (BrNANHS) and 4-sulfophenyl isothiocyanate (SPITC) maybe used. A method for selectively modifying α-amino groups with such a modification reagent is not particularly limited, and may be to, for example, Anal. Chem. 2007, 79, 7910-7915.

The ratio of the amount of the modification reagent used to the amount of a protein is 5-200:1, preferably 5-20:1 (on a molar basis).

The modification reaction may be carried out in a reaction system using, as a solvent, an aqueous solution or buffer solution containing an organic solvent selected from the group consisting of acetonitrile, tetrahydrofuran, dioxane, ethanol, methanol, isopropyl alcohol, and butanol. It is preferable that the pH of the solvent is adjusted to 6 to 10, preferably 7 to 9, more preferably 8 to 8.5.

As for the conditions of the modification reaction, the reaction temperature may be set to, for example, room temperature (e.g., 20 to 25° C.) to 60° C., and the reaction time may be set to, for example, 15 minutes to 6 hours.

The following scheme 2 shows an embodiment of the modification step in which TMPP modification of the C-terminal peptide fragment (A) and the other peptide fragments (B) is carried out using TMPP-Ac-OSu respectively to obtain a C-terminal peptide fragment modified (A′) and the other peptide fragments modified (B′). In the scheme 2, a group represented by TMPP is a tris(2,4,6-trimethoxyphenyl)phosphonium acetyl group.

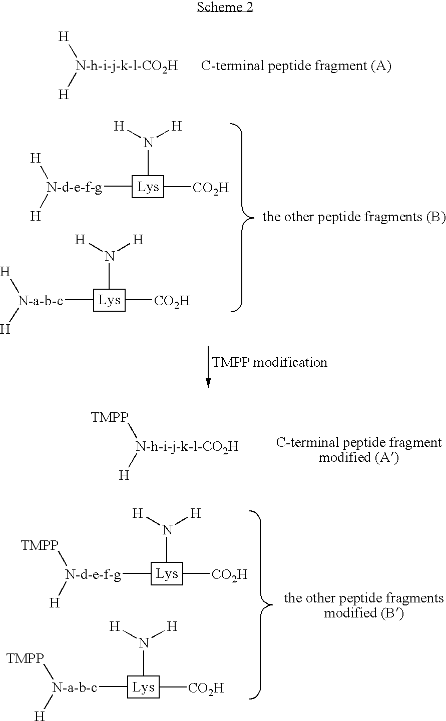

### <3. Separation Step>

The modified cleavage product containing the C-terminal peptide fragment modified (A′) and the other peptide fragments modified (B′) is subjected to a separation step. In the separation step, the C-terminal peptide fragment modified (A′) is separated from the other peptide fragments (B′).

A separation means is not particularly limited as long as it can hold via ε-amino group. More specifically, a carrier having a group capable of forming a covalent bond with an unsubstituted amino group (i.e., a free amino group) may be used.

Examples of a group capable of forming a covalent bond with an unsubstituted amino group include, but are not limited to, an isothiocyanate group, an imide group, an isourea group, an aldehyde group, a cyano group, an acetyl group, a succinyl group, a maleyl group, an acetoacetyl group, a dinitrophenyl group, and a trinitrobenzenesulfonic acid group. In the present invention, an isothiocyanate group is preferred, and a p-phenylenediisothiocyanate (DITC) group is particularly preferred.

The carrier part is not particularly limited, but may be made of, for example, a resin or glass. Specific examples thereof include silica gel, polystyrene, and porous glass.

In the modified cleavage product, the peptide fragments having an unsubstituted amino group correspond to the other peptide fragments modified (B′). Therefore, in the separation step, the other peptide fragments modified (B′) can be held to the separation means via their ε-amino groups. More specifically, the other peptide fragments modified (B′) may be reacted with the separation means so that covalent bonds are formed between them via their ε-amino groups. This makes it possible to allow only the other peptide fragments modified (B′) contained in the modified cleavage product to be held to the carrier, thereby allowing the C-terminal peptide fragment modified to be eluted. In this way, a C-terminal peptide of a protein of interest can be selectively recovered.

The following scheme 3 shows an embodiment of the separation step in which the cleavage product modified with TMPP is subjected to separation using a p-phenylenediisothiocyanate resin (DITC resin). As shown by the scheme 3, the other peptide fragments modified (B′) are covalently bonded to the DITC resin via their amino groups of lysine residues, while the C-terminal peptide fragment modified (A′) is not bonded to the DITC resin and can therefore be eluted.

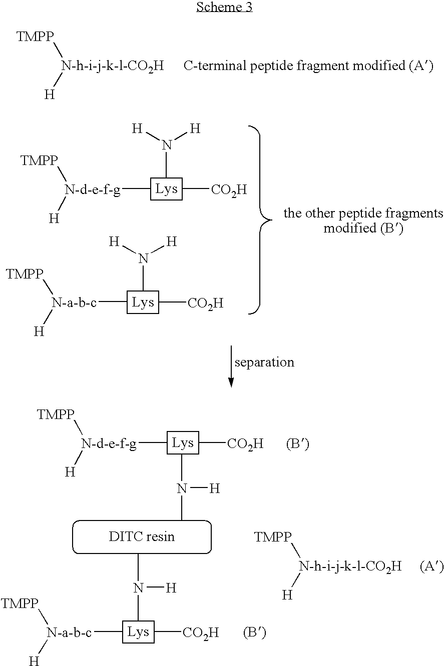

It is to be noted that according to a conventional method for recovering a C-terminal peptide, each of the N-terminal amino groups of the peptide fragments are bonded to a DITC resin. Therefore, in order to recover a C-terminal peptide fragment, it is necessary to cleave a bond between the C-terminal peptide fragment and the DITC resin using a strong acid such as TFA. In this case, however, the cleavage occurs on a peptide bond between an N-terminal amino acid residue of the C-terminal peptide fragment and its adjacent amino acid residue. Therefore, the N-terminal amino acid residue of the C-terminal peptide fragment remains bonded to the DITC resin, while the C-terminal peptide fragment which has lost its N-terminal amino acid residue is liberated from the DITC resin. Therefore, even when the recovered C-terminal peptide fragment is subjected to amino acid sequence analysis, its lost N-terminal amino acid cannot be identified.

### <4. Mass Spectrometry Step>

The recovered C-terminal peptide fragment has a modification group.

In a case where an electrically-charged modification reagent is used in the modification step described above, a C-terminal peptide fragment having an electrically-charged group bonded thereto is obtained. In this case, the electrically-charged group has the effect of enhancing the detection sensitivity of a C-terminal peptide fragment in mass spectrometry.

Particularly, in a case where an active ester of tris(2,4,6-trimethoxyphenyl)phosphonium acetic acid is used as a modification reagent in the modification step described above, a C-terminal peptide fragment having a strongly positively-charged TMPP group bonded thereto is recovered.

As described above, the method according to the present invention is excellent in that a recovered C-terminal peptide fragment has already given an extremely high detection sensitivity in mass spectrometry at the point that the C-terminal peptide is recovered. Further, the method according to the present invention is very advantageous in that de novo sequencing becomes possible by subjecting a C-terminal peptide fragment electrically charged by, for example, a TMPP group to mass spectrometry.

The amino acid sequence of the C-terminal peptide fragment may be determined by MS/MS analysis using a mass spectrometer based on ESI, PSD analysis using a MALDI-TOF mass spectrometer, or MS/MS analysis using a mass spectrometer based on MALDI.

### EXAMPLES

Hereinbelow, the present invention will be described in more detail with reference to the following example, but the present invention is not limited thereto.

### Experimental Example 1

### Study 1 Using Model Peptides

In this experimental example, a mixture of 4 kinds of model peptides was prepared, and was then subjected to the method according to the present invention using TMPP-Ac-OSu as a modification reagent.

More specifically, the following model peptides were used.

| | | |
|---|---|---|
| [1] | WAGGDASGE | (SEQ ID No. 1) |
| [2] | MHRQETVDCLK-NH₂ | (SEQ ID No. 2) |
| [3] | TRDIYETDYYRK | (SEQ ID No. 3) |
| [4] | AAKIQASFRGHMARKK | (SEQ ID No. 4) |

It is to be noted that a residue represented by K—NH₂ in the peptide [2] is a lysine residue whose C-terminal carboxyl group has been amidated.

The mixture of four kinds of model peptides corresponds to a protein cleavage product provided in the present invention. More specifically, the peptide [1] corresponds to a C-terminal peptide fragment, and the peptide fragments [2], [3], and [4] correspond to the other peptide fragments because they have a lysine residue as a C-terminal amino acid residue.

Equal amounts of the model peptides (100 pmol, 400 pmol in total) were mixed to obtain a model peptide mixture. The model peptide mixture was dissolved in 5 μL of a mixed solution of acetonitrile-water (volume ratio 1:9), and then 10 μL of a 50 mmol aqueous NaHCO₃ solution (pH 8.2) was added thereto to prepare a model peptide mixture solution.

The TMPP-Ac-Osu was prepared as a 1 mM solution using a mixed solution of acetonitrile-water (volume ratio 2:8) as a solvent.

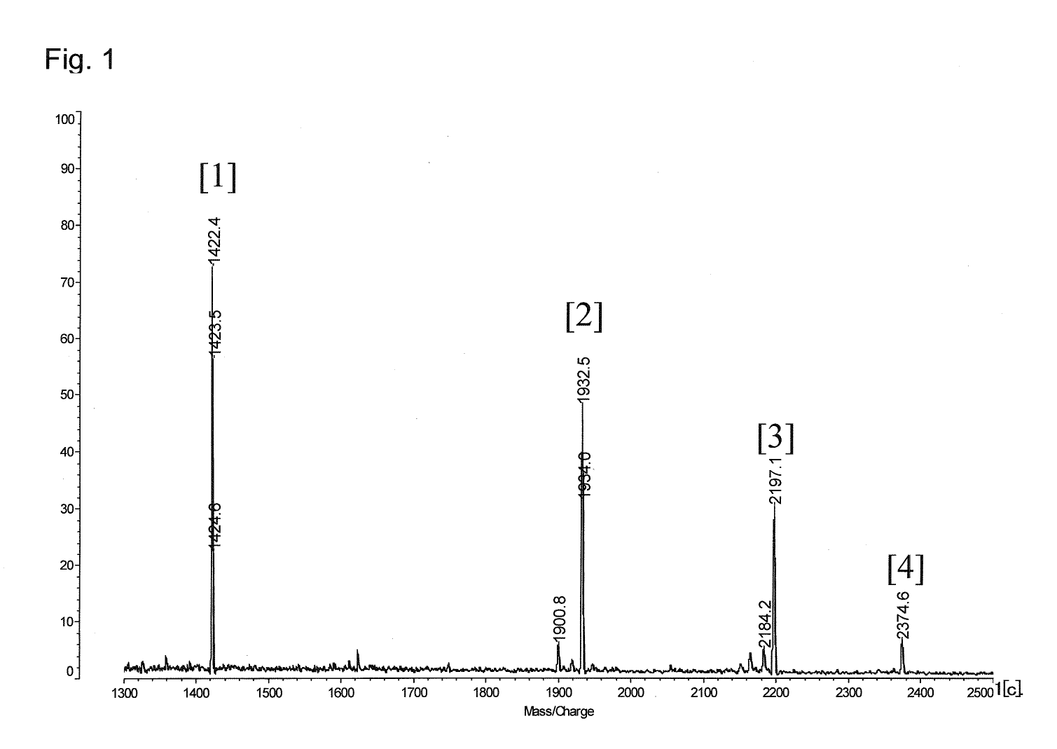

The thus prepared model peptide mixture solution was mixed with 5 μL of the 1 mM TMPP-Ac-OSu solution to react them with each other for 20 minutes in an ultrasonic water bath. The mass spectrum of the thus obtained reaction mixture is shown in FIG. 1 (horizontal axis: mass/charge, vertical axis: relative ion intensity, the same goes for the following mass spectra). As shown in FIG. 1, all the peptides modified with TMPP (indicated by numerals [1], [2], [3] and [4] in the mass spectrum) were observed.

5 mg of a DITC resin was washed with 100 μL of a mixed solution of 50 mmol aqueous NaHCO₃ solution (pH 8.2)-acetonitrile (volume ratio 9:1) prepared as a washing solution, and this washing was carried out twice.

The obtained reaction mixture was concentrated by centrifugation and dried to obtain a dry residue, and the dry residue was dissolved in 12 μL of a 50 mmol aqueous NaHCO₃ solution (pH 8.2). Then, 1 μL of the thus obtained solution (containing 8 pmol of each TMPP-modified peptide) was added to 5 mg of the washed DITC resin to react them with each other at 60° C. for 2 hours.

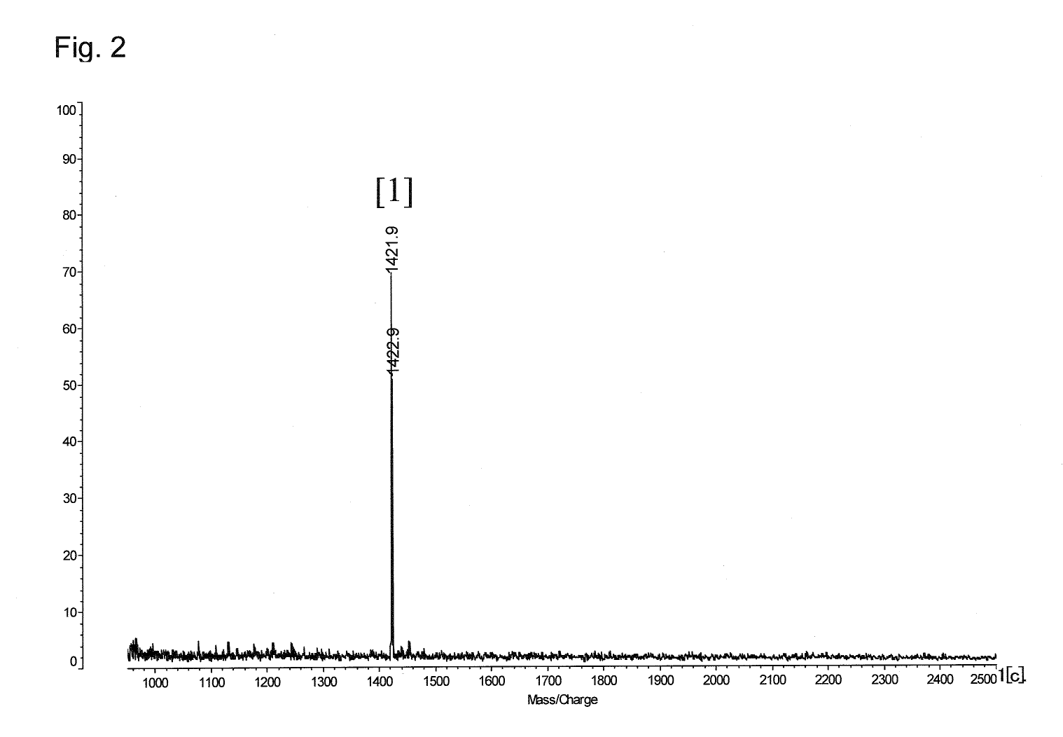

A mixed solution of acetonitrile-isopropyl alcohol-0.1 v/v % aqueous trifluoroacetic acid solution (volume ratio 1:1:2) was prepared as an elution solvent, and elution was carried out twice using 100 μL of the elution solvent. The thus obtained eluate was concentrated by centrifugation. The thus obtained dry residue was dissolved in 5 μL of a 0.1 v/v % aqueous trifluoroacetic acid solution, and was then subjected to mass spectrometry measurement. The thus obtained mass spectrum is shown in FIG. 2. As shown in FIG. 2, only the TMPP-modified peptide [1] corresponding to a C-terminal peptide fragment was observed. This result indicates that the selective recovery of the peptide [1] corresponding to a C-terminal peptide fragment was successfully performed.

### Example 1

### Study Using Protein

In this example, lysozyme (chick, egg-white) as a protein of interest was subjected to the method according to the present invention using TMPP-Ac-OSu as a modification reagent.

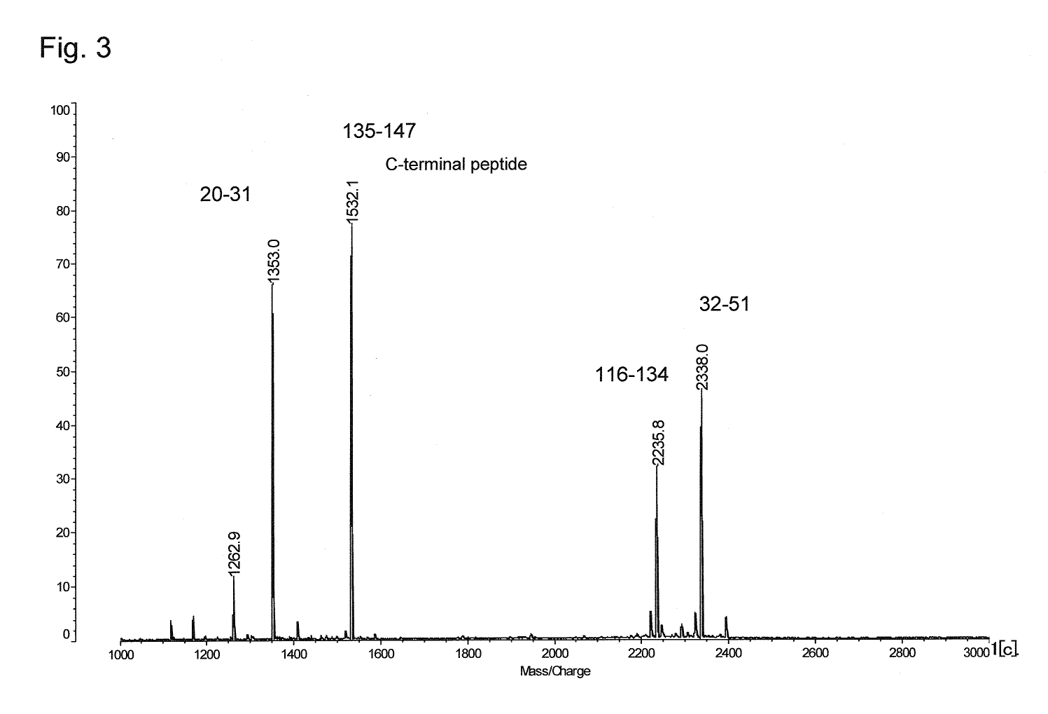

100 μg of a freeze-dried sample of lysozyme was dissolved in an aqueous solution containing 8 M urea and 50 mmol NaHCO₃, and then 1 μL of an aqueous TCEP solution (prepared by dissolving 5.7 mg of TCEP in 100 μL of water) was added thereto to react them with each other at 37° C. for 30 minutes. Then, 1 μL of an aqueous iodoacetamide solution (prepared by dissolving 9.3 mg of iodoacetamide in 100 μL of water) was added thereto to carry out an alkylation reaction at room temperature for 45 minutes. Then, 200 μL of a Lys-C solution (prepared by dissolving 5 μg of Lys-C in 200 μL of a 50 mmol aqueous NaHCO₃ solution) was added thereto to carry out a reaction at 37° C. overnight to digest the protein. The mass spectrum of a protein digest is shown in FIG. 3. As shown in FIG. 3, four peptide fragments (indicated by (20-31), (135-147), (116-134), and (32-51) in the mass spectrum) are strongly observed. Among these peptide fragments, the peptide fragment (135-147) is a C-terminal peptide fragment.

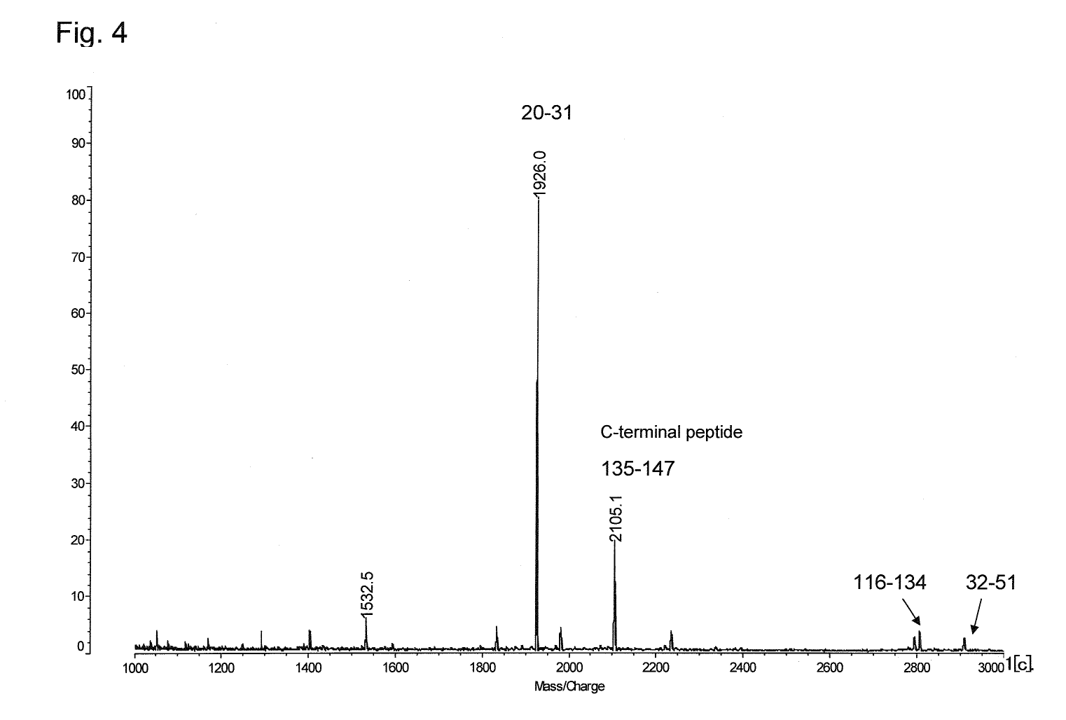

Then, 2 μL of the obtained protein digest solution (corresponding to 56 pmol of the lysozyme) was mixed with 10 μL of a 1 mmol aqueous TMPP-Ac-OSu solution to react them with each other for 20 minutes in an ultrasonic water bath. The mass spectrum of the thus obtained product is shown in FIG. 4. As can be seen from FIG. 4, it was confirmed that almost all the four kinds of the peptide fragments observed in FIG. 3 were modified with TMPP.

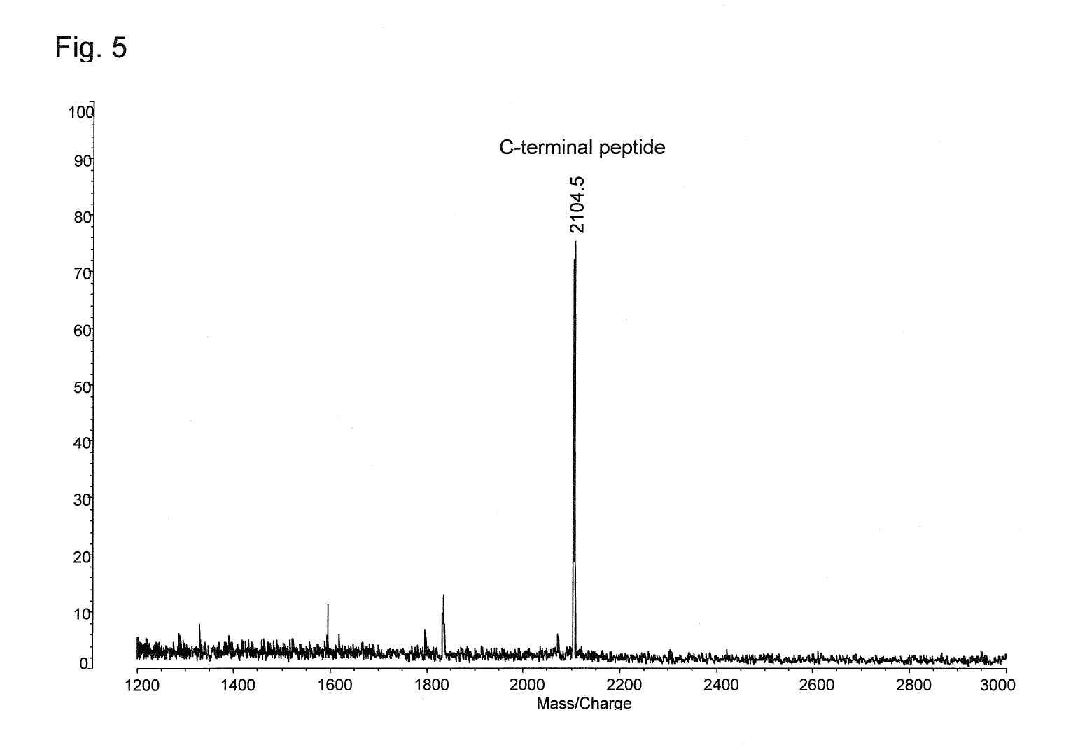

Then, 2 μL of the product (corresponding to 56 pmol of the lysozyme) obtained by TMPP modification was added to 5 mg of a washed DITC resin (prepared in the same manner as in Experimental Example 1) to react them with each other at 60° C. for 2 hours. After the completion of the reaction, extraction was carried out using an extraction solvent (prepared in the same manner as in Experimental Example 1). The thus obtained extract was concentrated and dried, and the obtained dry residue was dissolved in 10 μL of a 0.1 v/v % aqueous trifluoroacetic acid solution, and was then subjected to mass spectrometry measurement. The thus obtained mass spectrum is shown in FIG. 5. As can be seen from FIG. 5, it was confirmed that the C-terminal peptide fragment (135-147) was reliably isolated.

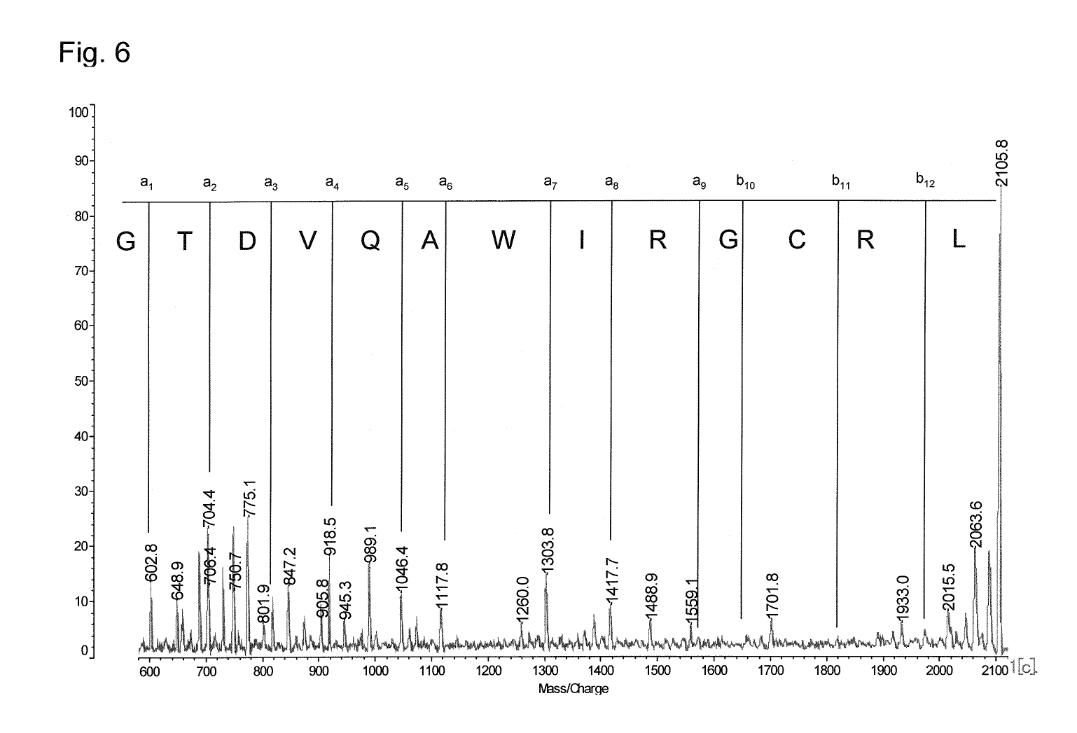

The sequence of the isolated peptide fragment was analyzed by MS/MS (CID). FIG. 6 shows the result of the amino acid sequence analysis. The peptide fragment isolated in Example 1 was ranked first also by MASCOT ion search.

### Experimental Example 2

### Study 2 Using Model Peptides

In order to determine the effect of improving fragmentation by modifying a side chain of an arginine residue, two kinds of peptides were each subjected to the method according to the present invention using TMPP-Ac-OSu as a modification reagent.

More specifically, the following model peptides were used.

| | | |
|---|---|---|
| [5] | RVYIHPF | (SEQ ID No. 5) |
| [6] | DAEFRHDSGYE | (SEQ ID No. 6) |

The model peptides [5] and [6] correspond to C-terminal peptide fragments contained in a protein cleavage product prepared using lysyl endopeptidase in the present invention.

The model peptide was dissolved in a mixed solution of 100 mM aqueous NaHCO₃ solution (pH 8.2)-acetonitrile (volume ratio 1:9) to prepare a 20 pmol/μL solution.

The TMPP-Ac-Osu was prepared as a 10 mM solution using a mixed solution of acetonitrile-water (volume ratio 2:8) as a solvent.

45 μL of the model peptide mixture solution was mixed with 5 μL of the 10 mM TMPP-Ac-Osu solution to react them with each other for 30 minutes in an ultrasonic water bath.

After the completion of TMPP modification, the thus obtained reaction mixture was mixed with 4 μL of 100 mM Na₂CO₃ and 6 μL of acetylacetone to react them with each other at 80° C. for 3 hours to modify a side chain of an arginine residue.

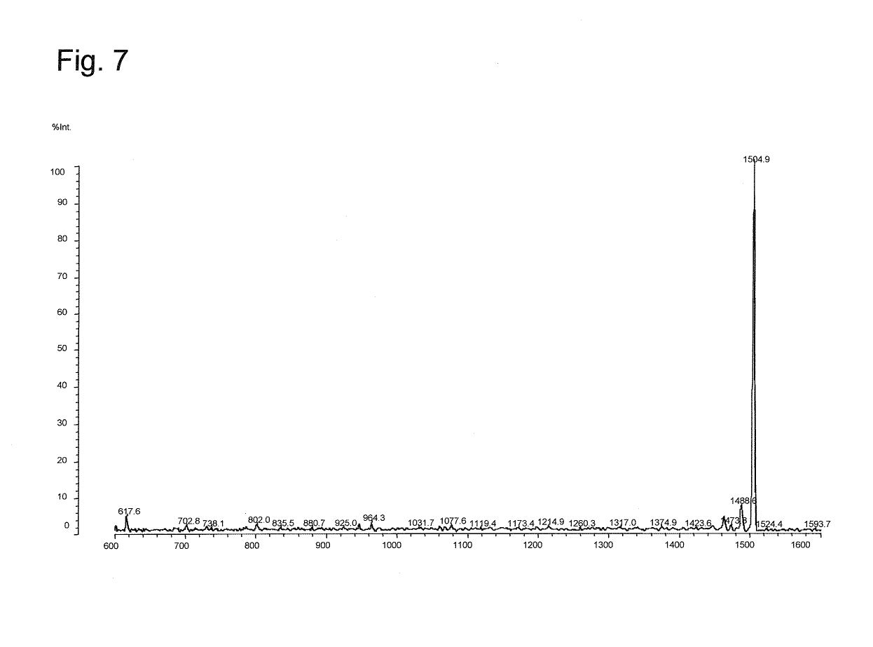

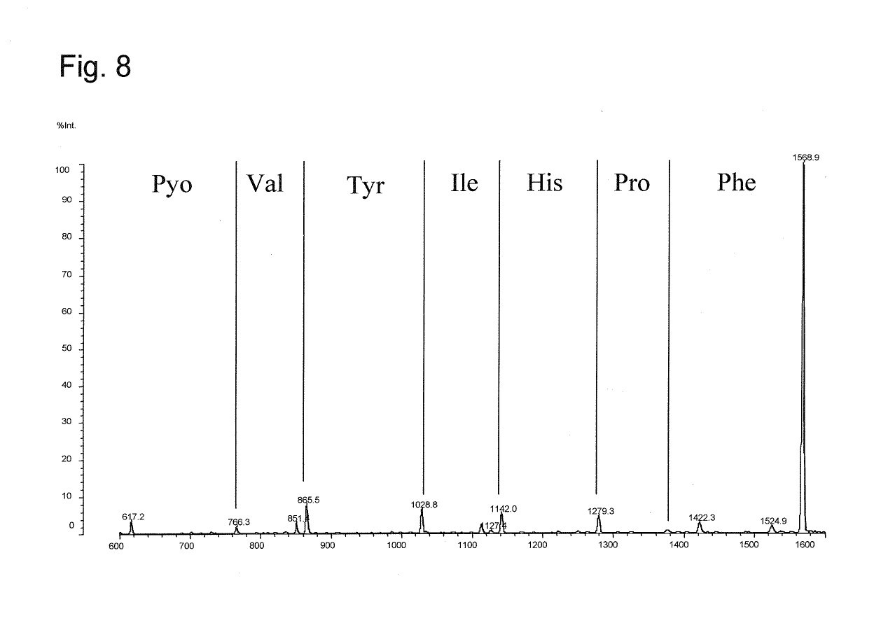

FIG. 7 shows the CID spectrum of the peptide [5] measured after TMPP modification, and FIG. 8 shows the CID spectrum of the peptide [5] measured after TMPP modification and arginine residue modification (horizontal axis: mass/charge, vertical axis: relative ion intensity, the same goes for the following spectra). In FIG. 7, no fragment ions were detected, and therefore the sequence of the peptide could not be determined. On the other hand, in FIG. 8, all the fragment ions were detected, and therefore the sequence of the peptide could be determined.

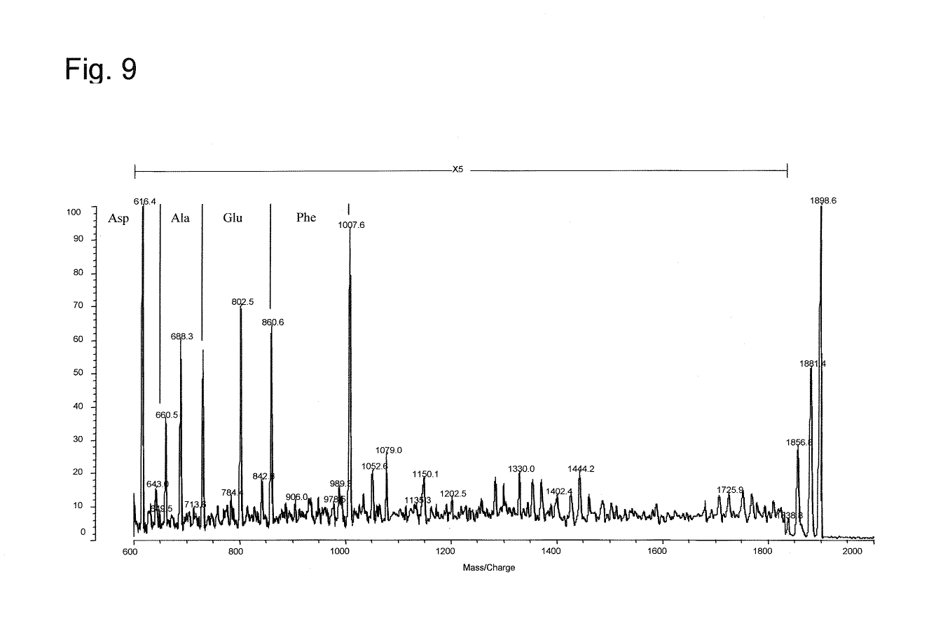

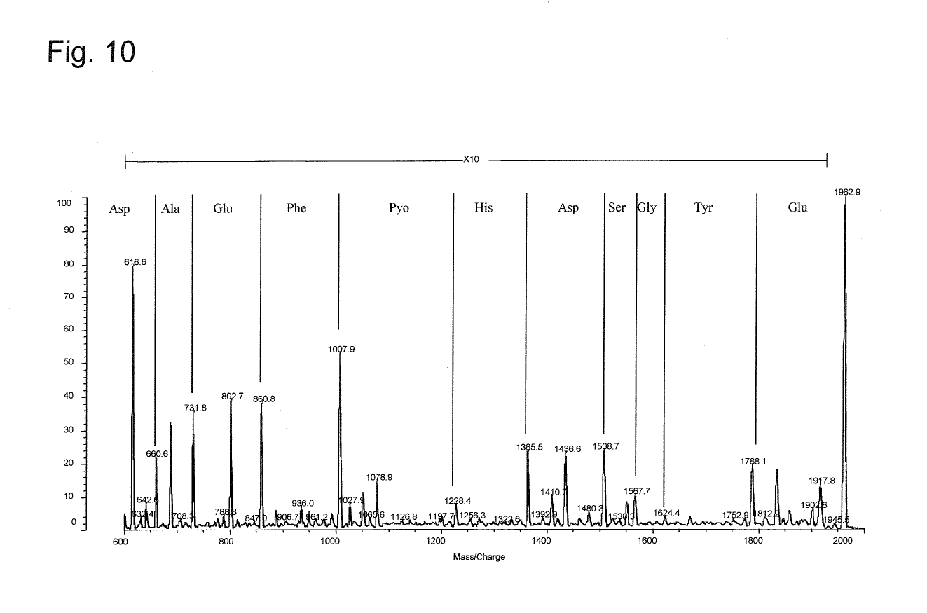

Further, FIG. 9 shows the CID spectrum of the peptide [6] measured after TMPP modification, and FIG. 10 shows the CID spectrum of the peptide [6] measured after TMPP modification and arginine residue modification. In FIG. 9, the peak of a fragment ion containing an arginine residue was very weak, and therefore the C-terminal sequence of the peptide could not be determined. On the other hand, in FIG. 10, all the fragment ions were detected, and therefore the sequence of the peptide could be determined. From the result, it has been confirmed that modification of a side chain of an arginine residue has the effect of improving fragmentation.

In the case of Example 1, it is possible to determine the sequence of an arginine residue-containing peptide contained in a measurement sample even when no particular modification of a side chain of an arginine residue is carried out. On the other hand, as shown in FIGS. 7 and 9 (Experimental Example 2), there is a case where the sequence of an arginine residue-containing peptide cannot be determined depending on the kind of sample. In this case, as shown in FIGS. 8 and 10, determination of the sequence of an arginine residue-containing peptide becomes possible by further modifying a side chain of an arginine residue.

Further, in Experimental Example 2, the reaction for modifying a side chain of an arginine residue was carried out under reaction conditions (i.e., at 80° C. for 3 hours) different from conventional reaction conditions (i.e., at room temperature for a dozen or so hours). It has been confirmed that when the modification of a side chain of an arginine residue is carried out under conventional reaction conditions, raw materials remain. On the other hand, when the modification of a side chain of an arginine residue was carried out under the reaction conditions employed in the Experimental Example, the reaction time was significantly reduced and no side reaction was observed. That is, the yield was enhanced and the reaction efficiency was significantly improved.

The example described above shows a concrete embodiment within the scope of the present invention, but the present invention is not limited to the example and can be implemented in various embodiments. Therefore, the example described above is merely illustrative in every respect, and should not be construed as being restrictive. Further, the changes that fall within the equivalents of the claims are all within the scope of the present invention.

What is claimed is:

1. A method for selectively recovering a C-terminal peptide of a protein, comprising the steps of:
providing a cleavage product of a protein of interest containing a C-terminal peptide fragment (A) having an α-amino group but not having an ε-amino group and the other peptide fragments (B) having an α-amino group and an ε-amino group;
selectively modifying the α-amino groups in the cleavage product of the protein of interest with a modification reagent to obtain a modified cleavage product containing a C-terminal peptide fragment modified (A′) having a modified amino group but not having the ε-amino group and the other peptide fragments modified (B′) having a modified amino group and the ε-amino group; and
separating the C-terminal peptide fragment modified (A′) from the modified cleavage product by allowing a carrier to hold the other peptide fragments modified (B′) via the -amino group.

2. The method for selectively recovering a C-terminal peptide of a protein according to claim 1, wherein the modification reagent is positively-charged.

3. The method for selectively recovering a C-terminal peptide of a protein according to claim 1, wherein the modification reagent is selected from the group consisting of tris(2,4,6-trimethoxyphenyl)phosphonium acetic acid and derivatives thereof.

4. The method for selectively recovering a C-terminal peptide of a protein according to claim 1, wherein the modification reagent is selected from the group consisting of a tris(2,4,6-trimethoxyphenyl)phosphonium acetic acid N-hydroxysuccinimide ester and a tris(2,4,6-trimethoxyphenyl)phosphonium acetic acid sulfosuccinimide ester.

5. The method for selectively recovering a C-terminal peptide of a protein according to claim 1, wherein the cleavage product of the protein of interest is obtained by digesting a protein of interest with lysyl endopeptidase.

6. The method for selectively recovering a C-terminal peptide of a protein according to claim 1, further comprising the step of chemically modifying a side chain of an arginine residue that can be contained in the cleavage product of the protein of interest, the modified cleavage product, or the separated C-terminal peptide fragment (A′).

7. The method for selectively recovering a C-terminal peptide of a protein according to claim 1, wherein the carrier has p-phenylenediisothiocyanate immobilized thereto.

8. A method for determining the amino acid sequence of a C-terminal peptide of a protein, comprising the steps of:
selectively recovering a C-terminal peptide of a protein of interest by the method according to claim 1; and
determining the amino acid sequence of the C-terminal peptide by subjecting a recovered C-terminal peptide fragment to mass spectrometry measurement.
